(19) United States
(12) Patent Application Publication
(10) Pub. No.: US 2010/0004315 A1
(43) Pub. Date: Jan. 7, 2010

(54) Biodegradable Cross-Linked Branched Poly(Alkylene Imines)

(76) Inventors: Gregory Slobodkin, Huntsville, AL (US); Majed Matar, Madison, AL (US); Brian Jeffery Sparks, Huntsville, AL (US); Jason Fewell, Madison, AL (US); Khursheed Anwer, Madison, AL (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/404,989

(22) Filed: Mar. 16, 2009

Related U.S. Application Data
(60) Provisional application No. 61/036,775, filed on Mar. 14, 2008.

(51) Int. Cl. C08G 73/04; A61K 31/7105; A61K 31/7052

(52) U.S. Cl. 514/44 A; 525/417; 525/54.1; 514/44 R; 514/43

(57) ABSTRACT

Disclosed is a cross-linked branched poly(alkylenimine) and compositions thereof and nucleotide molecules. Also disclosed are methods for preparing the cross-linked branched poly(alkylenimine).

### CROSS-REFERENCE TO RELATED APPLICATIONS

This application claims the benefit of Provisional Application No. 61/036,775, filed Mar. 14, 2008, which is incorporated herein by reference in its entirety.

### FIELD OF THE INVENTION

The present invention relates to cross-linked polymers, to pharmaceutical compositions thereof, and to methods of using and preparing the cross-linked polymers and compositions.

### DESCRIPTION OF THE RELATED ART

The success of gene therapy relies on the ability of gene delivery systems to efficiently and safely deliver the therapeutic gene to the target tissue. Gene delivery systems can be divided into viral and non-viral (or plasmid DNA-based). The present gene delivery technologies being used in clinics today can be considered first generation, in that they possess the ability to transfect or infect target cells through their inherent chemical, biochemical, and molecular biological properties. Relying on these sole properties, however, limits therapeutic applications. For example, viruses with the ability to infect mammalian cells have been effectively used for gene transfer with high transduction efficiency. However, serious safety concerns (e.g., strong immune response by the host and potential for mutagenesis) have been raised when viral systems have been used in clinical applications.

The non-viral gene delivery systems, based on “naked DNA” or formulated plasmid DNA, have potential benefits over viral vectors due to simplicity of use and lack of inciting a specific immune response. A number of synthetic gene delivery systems have been described to overcome the limitations of naked DNA, including cationic lipids, peptides, and polymers. Despite early optimism, the clinical relevance of the cationic lipid-based systems is limited due to their low efficiency, toxicity, and refractory-nature.

Polymers, on the other hand, have emerged as a viable alternative to current systems because their excellent molecular flexibility allows for complex modifications and incorporation of novel chemistries. Cationic polymers, such as poly(L-lysine) (PLL) and poly(L-arginine) (PLA), polyethyleneimine (PEI) have been widely studied as gene delivery candidates due to their ability to condense DNA, and promote DNA stability and transmembrane delivery. The transfection efficiency of the cationic polymers is influenced by their molecular weight. Polymers of high (>20 kD) molecular weight have better transfection efficiency than polymers of lower molecular weight. However, polymers with high molecular weights are also more cytotoxic. Several attempts have been made to circumvent this problem and improve the transfection activity of cationic polymers without increasing their cytotoxicity. For example, Lim et al. have synthesized a degradable polymer, poly [α-(4-aminobutyl)-L-glycolic acid] (PAGA) by melting condensation. Pharm. Res. 17:811-816, 2000. Although PAGA has been used in some gene delivery studies, its practical application is limited due to low transfection activity and poor stability in aqueous solutions. J Controlled. Rel. 88:33-342, 2003; Gene Ther. 9:1075-1084, 2002. Hydroxyproline ester (PHP ester) and networked poly(amino ester) are among a few other examples of degradable polymers. The PHP ester has been synthesized from Cbz-4-hydroxy-L-proline by melting condensation or by dicyclohexylcarbodiimide (dimethyl-amino)pyridine (DCC/DMAP)-activated polycondensation. J. Am. Chem. Soc. 121:5633-5639, 1999; Macromolecules 32:3658-3662, 1999. The networked poly(amino ester) (n-PAE) has been synthesized using bulk polycondensation between hydroxyl groups and carboxyl groups of bis(2-methoxy-carbonylethyl)[tris-(hydroxymethyl)methyl]amine followed by condensation with 6-(Fmoc-amino)hexanoic acid (Bioconjugate Chem.13:952-957, 2002). These polyesters have been shown to condense DNA and transfect cells in vitro with low cytotoxicity, but their stability in aqueous solutions is poor.

### SUMMARY OF THE INVENTION

In one aspect, the invention provides intermolecularly cross-linked poly(alkylene imines) consisting of branched poly(alkylene imine) units having primary, secondary and tertiary amino groups, the units being covalently cross-linked to one another by primary amino groups in the poly(alkylene imine) units and short chain linkers having a biodegradable bond, where at least one primary amino nitrogen is optionally protected, and at least one unit is optionally bonded to a targeting ligand, a visualizing agent, and/or a lipophilic group.

In another aspect, the invention provides compounds which are branched poly(alkylene imines) having substantially all of the primary amino nitrogen atoms protected by first protecting groups, and substantially all of the secondary amino nitrogen atoms protected by second protecting groups.

In another aspect, the invention provides compounds which are branched poly(alkylene imine) having substantially all of its primary amino nitrogen atoms unprotected and substantially all of its secondary amino nitrogen atoms protected.

In yet another aspect, the invention provides a compound which is branched poly(alkylene imine) having a plurality of primary and secondary nitrogen atoms, wherein

(a) substantially all of the secondary amino nitrogen atoms are protected by protecting groups;

(b) the primary amino nitrogen atoms are
(i) unprotected; or
(ii) protected; or
(iii) bonded to R₁, where R₁ is a lipophilic group, a targeting ligand, and/or a visualizing agent; and
at least one of the primary nitrogens is protected, and at least one of the primary nitrogen atoms is bonded to R₁.

In still another aspect, the invention provides pharmaceutical compositions comprising a cross-linked poly(alkylene imine) of the invention and nucleotide molecule. In certain aspects, the nucleotide is a small RNA molecule.

The invention further provides processes for making the cross-linked poly(alkylene imines) of the invention. The processes comprise (a) reversibly blocking at least about 50% of secondary nitrogen atoms within branched poly(alkylenimine) to form protected branched poly(alkylenimine); and (b) cross-linking the protected branched poly(alkylenimine) with a short-chain linker having a biodegradable bond. If desired, the protected branched poly(alkylenimine) units may be deprotected following cross-linking.

In yet another aspect, the invention provides other processes for preparing the cross-linked poly(alkylene imines) of the invention. These processes comprise (a) reversibly blocking at least about 75% of the primary nitrogen atoms within branched poly(alkylene imine) to form a primary-nitrogen protected branched poly(alkylenimine); (b) reversibly blocking at least about 50% of secondary nitrogen atoms within the primary-nitrogen branched poly(alkylenimine) to form primary-nitrogen and secondary-nitrogen protected branched poly(alkylenimine); (c) deprotecting the primary nitrogen atoms in the primary-nitrogen and secondary-nitrogen protected branched poly(alkylenimine) to produce secondary-nitrogen protected branched poly(alkylenimine); and (d) cross-linking the secondary-nitrogen protected branched poly(alkylenimine) with a short-chain linker having a biodegradable bond to form a secondary-nitrogen protected cross-linked branched poly(alkylenimine). If desired, the secondary nitrogens in the cross-linked branched poly(alkylene imine) can be deprotected following cross-linking.

Also, if desired, the cross-linked branched poly(alkylene imines) may be further modified to carry a targeting ligand, a visualizing agent, and/or a lipophilic group; typically by reacting protected precursors with such appropriate reagents prior to cross-linking.

The invention further provides cross-linked branched poly(alkylene imines) that are produced by the processes of the invention.

When in aqueous media in formulations at physiological pH or lower, the cross-linked branched poly(alkylene imines) of the invention generally exist in the cationic form. In other words, some of the available nitrogen atoms will be in cationic, i.e., protonated form.

### BRIEF DESCRIPTION OF THE DRAWINGS

FIG. 1 shows results of electrophoresis demonstrating complexation between siRNA and a polymer according to another aspect of the present invention;

FIGS. 2A and 2B show graphs of data describing GAPDH or Luciferase activity compared against appropriate controls according to yet another aspect of the present invention;

FIG. 3 shows a graph of data describing VEGF expression of siRNA complexes prepared with branched PEI based cross-linked polymer as compared to control siRNA complexes according to a further aspect of the present invention;

FIG. 4 shows a graph of data describing VEGF expression of siRNA complexes prepared with branched PEI based cross-linked polymer as compared to control siRNA complexes according to yet a further aspect of the present invention;

FIG. 5 shows a graph of data describing inhibition of ApoB transcript with siRNA complexes prepared with branched PEI based cross-linked polymer as compared to the control siRNA complexes according to another aspect of the present invention; and

FIGS. 6A and 6B show graphs of data describing expression of GAPDH in lung and liver tissue of mice following IV injection of GAPDH siRNA formulated with a cross-linked branched poly(alkylene imine) of the invention as compared to GAPDH levels in control mice that have been injected with formulated non silencing siRNA according to yet another aspect of the present invention.

FIG. 8. is a graph of the data describing VEGF transcript levels in lung and spleen of mice following iv injection of VEGF siRNA formulated with a cross-linked branched poly(alkylene imine) of the invention and formulated non-silencing siRNA.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and “the” include plural referents unless the context clearly dictates otherwise. Thus, for example, reference to a polymer containing “a molecule” includes reference to a polymer having one or more of such molecules, and reference to “an antibody” includes reference to one or more of such antibodies.

### DEFINITIONS

In describing and claiming the present invention, the following terminology will be used in accordance with the definitions set out below.

As used herein, the terms “transfecting” and “transfection” refer to the transportation of nucleic acids from the environment external to a cell to the internal cellular environment, with particular reference to the cytoplasm and/or cell nucleus. Without being bound by any particular theory, it is to be understood that nucleic acids may be delivered to cells either after being encapsulated within or adhering to polymer complexes or being entrained therewith. Particular transfecting instances deliver a nucleic acid to a cell nucleus.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “composition” refers to a mixture of two or more compounds, elements, or molecules. In some aspects the term “composition” may be used to refer to a mixture of a nucleic acid and a delivery system.

As used herein, “small” when used in reference to a nucleotide sequence refers to a nucleotide sequences having a nucleotide chain length of about 17-30 base pairs in one aspect, or 10-100 base pairs in another aspect.

As used herein, the terms “administration,” “administering,” and “delivering” refer to the manner in which a composition is presented to a subject. Administration can be accomplished by various art-known routes such as oral, parenteral, transdermal, inhalation, and implantation. Thus, an oral administration can be achieved by swallowing, chewing, sucking of an oral dosage form comprising the composition.

Parenteral administration can be achieved by injecting a composition intravenously, intra-arterially, intramuscularly, intraarticularly, intrathecally, intraperitoneally, subcutaneously, intratumorally, and intracranially.

Injectables for such use can be prepared in conventional forms, either as a liquid solution or suspension, or in a solid form that is suitable for preparation as a solution or suspension in a liquid prior to injection, or as and emulsion. Additionally, transdermal administration can be accomplished by applying, pasting, rolling, attaching, pouring, pressing, and rubbing of a transdermal composition onto a skin surface. These and additional methods of administration are well-known in the art. Suitable excipients that can be used for administration include, for example, water, saline, dextrose, glycerol, ethanol, and the like; and if desired, minor amounts of auxiliary substances such as wetting or emulsifying agents, buffers, and the like.

As used herein, the terms “nucleotide sequence” and “nucleic acids” may be used interchangeably, and refer to DNA and RNA, as well as synthetic congeners thereof. Non-limiting examples of nucleic acids may include plasmid DNA encoding protein or inhibitory RNA producing nucleotide sequences, synthetic sequences of single or double strands, missense, antisense, nonsense, as well as on and off and rate regulatory nucleotides that control protein, peptide, and nucleic acid production. Additionally, nucleic acids may also include, without limitation, genomic DNA, cDNA, RNAi, siRNA, shRNA, mRNA, tRNA, rRNA, microRNA, and hybrid sequences or synthetic or semi-synthetic sequences. Additionally, nucleic acids may be of natural or artificial origin, or both. In one aspect, a nucleotide sequence may also include those encoding for synthesis or inhibition of a therapeutic protein. Non-limiting examples of such therapeutic proteins may include anti-cancer agents, growth factors, hypoglycemic agents, anti-angiogenic agents, bacterial antigens, viral antigens, tumor antigens or metabolic enzymes. Examples of anti-cancer agents may include interleukin-2, interleukin-4, interleukin-7, interleukin-12, interleukin-15, interferon-α, interferon-β, interferon-γ, colony stimulating factor, granulocyte-macrophage stimulating factor, anti-angiogenic agents, tumor suppressor genes, thymidine kinase, eNOS, iNOS, p53, p16, TNF-α, Fas-ligand, mutated oncogenes, tumor antigens, viral antigens or bacterial antigens. In another aspect, plasmid DNA may encode for an RNAi molecule designed to inhibit protein(s) involved in the growth or maintenance of tumor cells or other hyperproliferative cells. Furthermore, in some aspects a plasmid DNA may simultaneously encode for a therapeutic protein and one or more RNAi molecules. In other aspects a nucleic acid may also be a mixture of plasmid DNA and synthetic RNA, including sense RNA, antisense RNA, and ribozymes. In addition, the nucleic acid can be variable in size, ranging from oligonucleotides to chromosomes. These nucleic acids may be of human, animal, vegetable, bacterial, viral, or synthetic origin. They may be obtained by any technique known to a person skilled in the art.

As used herein, the term “peptide” may be used to refer to a natural or synthetic molecule comprising two or more amino acids linked by the carboxyl group of one amino acid to the alpha amino group of another. A peptide of the present invention is not limited by length, and thus “peptide” can include polypeptides and proteins. Non-limiting examples of peptides that can be beneficial include oxytocin, vasopressin, adrenocorticotrophic hormone, epidermal growth factor, prolactin, luliberin or luteinising hormone releasing hormone, growth hormone, growth hormone releasing factor, insulin, somatostatin, glucagon, interferon, gastrin, tetragastrin, pentagastrin, urogastroine, secretin, calcitonin, enkephalins, endorphins, angiotensins, renin, bradykinin, bacitracins, polymixins, colistins, tyrocidin, gramicidines, and synthetic analogues, modifications and pharmacologically active fragments thereof, as well as monoclonal antibodies and soluble vaccines.

As used herein, the terms “covalent” and “covalently” refer to chemical bonds whereby electrons are shared between pairs of atoms.

As used herein, “drug,” “active agent,” “bioactive agent,” “pharmaceutically active agent,” “drug,” and “pharmaceutical,” may be used interchangeably, and refer to an agent or substance that has measurable specified or selected physiologic activity when administered to a subject in a significant or effective amount. These terms of art are well-known in the pharmaceutical and medicinal arts. Examples of such substances include broad classes of compounds that can be delivered to the subject. In general, this includes, but is not limited to: nucleic acids and oligonucleotides; anti-infectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium, calcium channel blockers, beta-blockers, alpha-blockers, and antiarrhythmics; antihypertensives; diuretics and antidiuretics; vasodilators including general, coronary, peripheral and cerebral; central nervous system stimulants; vasoconstrictors; cough and cold preparations, including decongestants; hormones such as estradiol and other steroids including corticosteroids; hypnotics; immunosuppressives; muscle relaxants; parasympatholytics; psychostimulants; sedatives; and tranquilizers. By the method of the present invention, drugs in all forms, e.g. ionized, nonionized, free base, acid addition salt, and the like may be delivered, as can drugs of either high or low molecular weight.

As used herein, the term “biodegradable” refers to the conversion of materials into less complex intermediates or end products by solubilization hydrolysis, reduction, or by the action of biologically formed entities which can be enzymes and other products of the organism.

As used herein, the term “polymeric backbone” is used to refer to a collection of polymeric backbone molecules having a weight average molecular weight within a designated range. A polymeric backbone generally has at least two terminal ends of the molecule. In the case of a branched polymeric backbone, for example, each branch would be considered to have at least one terminal end.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “unit” when used in reference branched poly(alkylene imine) (BPAI) refers to a molecule of a branched poly(alkylene imine) polymer, prior to cross-linking. The units of BPAI may carry visualizing agents or other groups as discussed herein; such groups can be incorporated into the BPAI as desired prior to cross-linking.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum.

Furthermore, such an interpretation should apply regardless of the breadth of the range or the characteristics being described.

Fundamental to the success of gene therapy is the development of gene delivery vehicles that are safe and efficacious after systemic administration. The invention provides an efficient non-viral polymer-based gene carrier for delivery and/or expression of nucleic acids to a target cell.

In one aspect, for example, a polymeric nucleotide expression composition is provided including a biodegradable, cross-linked branched poly(alkylene imine), wherein the branched poly(alkylenimine) units are cross-linked together by a short chain linker having a biodegradable bond. The composition further includes a nucleotide sequence associated with the biodegradable cross-linked poly(alkylene imine). In some aspects, the compositions of the present invention are particularly suited for the delivery of small nucleotide sequences. As noted above, when in aqueous media in formulations at physiological pH or lower, the cross-linked branched poly(alkylene imines) of the invention generally exist in the cationic form. Thus, preferred polymeric nucleotide expression compositions of the invention are considered cationic because some of the available nitrogen atoms in the biodegradable cross-linked poly(alkylene imine) will be in protonated form.

A variety of nucleotide sequences may be associated with the polymeric vehicles of the present invention. Although such nucleotide sequences may include larger nucleotide macromolecules, the polymeric system is particularly useful for the delivery and expression of small nucleotide sequences. In one aspect such small nucleotide sequences may include, without limitation, RNAi, siRNA, shRNA, mRNA, tRNA, rRNA, and microRNA. In one specific aspect, the small nucleotide sequence may include siRNA. As is shown in the examples below, the polymeric vehicle is surprisingly well suited for the delivery and/or expression of RNAi moieties such as siRNA. The molar ratio of nitrogen in the poly(alkylene imine) units to phosphate in the nucleotide molecule is from about 5:1 to about 200:1, preferably from about 10:1 to about 100:1, and more preferably from about 20:1 to about 50:1.

In still another aspect, the invention provides pharmaceutical compositions comprising a cross-linked poly(alkylene imine) of the invention and nucleotide molecule. In certain aspects, the nucleotide is a small RNA molecule. In these compositions, the nucleotide molecule is associated with the cross-linked poly(alkylene imine). The nucleotide molecule in the compositions is selected from the group consisting of siRNA, shRNA, dsRNA, ssRNA, mRNA, rRNA, microRNA, DNA, plasmids, cDNA, and combinations thereof.

The compositions may further comprise a coformulant selected from the group consisting of dioleoyl phosphatidylethanolamine, cholesterol, galactosylated lipid, polyethyleneglycol-conjugated lipid, and combinations thereof.

The polymeric gene expression compositions of the present invention may optionally include a functional moiety covalently coupled to the branched poly(alkylenimine) copolymer. Non-limiting examples of such functional moieties includes visualizing agents such as fluorescent markers; lipids; fatty acids; receptor ligands; membrane permeating agents; endosomolytic agents; nuclear localization sequences; and pH sensitive endosomolytic peptides. In one aspect, the functional moiety can be a fatty acid including a member selected from the group consisting of butyric acid, caproic acid, caprylic acid, capric acid, lauric acid, myristic acid, palmitic acid, stearic acid, myristoleic acid, palmitoleic acid, oleic acid, linoleic acid, alpha-linolenic acid, and combinations thereof. Where employed, the visualizing agents can be incorporated into the cross-linked biodegradable branched poly(alkylene imines) of the invention at a degree of about 0.01 to 0.2, preferably about 0.07 to 0.15, most preferably about 0.09 to 0.11 mole of visualizing agent per mole of branched poly(alkylene imine) unit, or a degree of about 0.05 to 1, more preferably about 0.15 to 0.4, most preferably about 0.25 to 0.35 moles of visualizing agent per mole of the cross-linked polymer.

The present invention additionally provides a polymeric nucleotide expression composition including a biodegradable cross-linked branched poly(alkylenimine), wherein the branched poly(alkylenimine) units are cross-linked together by a short chain linker having a biodegradable bond, and a nucleotide molecule associated with the biodegradable cross-linked branched poly(alkylenimine). Non-limiting examples of nucleotide molecules may include siRNA, shRNA, microRNA, dsRNA, ssRNA, mRNA, rRNA, DNA, plasmids, cDNA, and combinations thereof.

The present invention further provides a method for making a biodegradable cross-linked branched poly(alkylenimine), wherein the branched poly(alkylenimine) units are cross-linked together by a short chain linker with a biodegradable bond. Such a method may include blocking reversibly at least 50% of primary and secondary nitrogen atoms of a plurality of branched poly(alkylenimine) units to form protected branched poly(alkylenimine) units, cross-linking the plurality of protected branched poly(alkylenimine) units with a linker having a biodegradable bond, and deprotecting the protected branched poly(alkylenimine) units following cross-linking. This method of blocking-reacting-deprotecting allows for the addition of any ligands.

Various polyalkylenimines are contemplated for use in aspects of the invention as polymeric backbones for nucleotide delivery and/or expression. Non-limiting examples of suitable poly(alkylene imines) are poly(trimethyleneimine), poly(tetraethyleneimine), poly(1,2-propyleneimine), poly(ethyleneimine), and combinations thereof. In a particular aspect of the invention the branched poly(alkyleneimine) is a branched poly(ethyleneimine) (“BPEI”, “PEI”, or “branched PEI”). A preferred branched PEI for use herein has a molecular weight of from about 1000 daltons to about 4000 daltons, more preferably from about 1200 to 2500 daltons, and most preferably from about 1500 to 2000 daltons.

PEI efficiently condenses DNA into small narrowly distributed positively charged spherical complexes, and can transfect cells in vitro and in vivo. PEI is similar to other cationic polymers in that the transfection activity of PEI increases with increasing polymer/DNA ratios. A distinct advantage of PEI over PLL is its endosomolytic activity which enables PEI to yield high transfection efficiency. A branched PEI suitable for use herein has about 25% primary nitrogen atoms, about 50% secondary nitrogen atoms, and about 25% tertiary nitrogen atoms.

The overall degree of protonation of PEI in aqueous media doubles from pH 7 to pH 5, which means in the endosome PEI becomes heavily protonated. Without intending to be bound by any theory, it is believed that protonation of PEI triggers chloride influx across the endosomal membrane, and water follows in to counter the high ion concentration inside the endosome, which eventually leads to endosomal disruption from osmotic swelling and release of the entrapped DNA. Because of its intrinsic endosomolytic activity, PEI generally does not require the addition of an endosomolytic agent for transfection. Additionally, the cytotoxicity and transfection activity of PEI is more or less linearly related to the molecular weight of the polymer.

The use of free BPEIs may present certain inconveniences due to hygroscopicity as anhydrous free bases or as salts such as chloride, and to cytotoxicity observed with higher molecular weight BPEIs. The invention aims at the bypassing or mitigating the high MW BPEI cytotoxicity by assembling a larger MW biodegradable aggregate from smaller BPEI units. Any bifunctional linker used for PEI cross-linking can form a link either between two nitrogen atoms belonging to the same polymer unit (i.e. forming a loop without actually linking polymer molecules) or between two nitrogen atoms from different polymer units (i.e. truly linking polymer units). Since it can be difficult to distinguish between these two modes of linkage spectroscopically, one useful analytical test would be determination of molecular weight by light scattering or solution viscosity measurements and determination of the biological activity of the resulting cross-linked product (See for example J. Mater. Chem. 1995, 5, 405-411, which is incorporated herein by reference). In the vicinity of any given nitrogen atom the local concentration of the same-backbone nitrogens is high and not dependent on the solution concentration, while the concentration of the nitrogens from different backbones is low and concentration dependent. Therefore, under normal conditions, loop formation can be expected to be the preferred reaction pathway for the linker.

In order to minimize such loop formation, at least one of the following approaches can be utilized. The first approach may include increasing the concentration of the polymer molecules in the reaction mixture. The second approach may include decreasing the number of available nitrogen atoms on every polymer molecule by reversibly blocking these nitrogen atoms with suitable protecting groups. At the limit, with only one nitrogen atom available per molecule, loop formation becomes impossible and the only possible aggregate is a dimer. For less exhaustively protected polymers, the local concentration of nitrogen atoms from other polymer chains declines in parallel with that of the same-chain nitrogens but can be made comparable to it, leading to a 50% chance of linking vs. loop formation. Although molecular weights may vary depending on a variety of factors, in one aspect the molecular weights of cross-linked polymers may range from about 15,000 Da to about 25,000 Da. In another aspect the molecular weights of cross-linked polymers may range from about 3,000 Da to about 10,000 Da. In yet another aspect the molecular weights of cross-linked polymers may range from about 500 Da to about 2,000 Da. In a further aspect, the molecular weights of cross-linked polymers may range from about 500 Da to about 25,000 Da.

In one aspect, suitable cross-linking BPEI aminogroups include primary aminogroups on or near the surface of the BPEI molecule. Therefore, in the case of BPEIS, the aforementioned protection should be chemoselective, protecting all, or almost all, of the secondary aminogroups while leaving a portion of the primary aminogroups free.

BPEIs can be converted into protected forms using tert-butoxycarbonyl (BOC) as a protecting group during assembly of the BPEI aggregate. These reactions are typically carried out in the absence of water, i.e., in an organic solvent. In one aspect, about 50% to about 99% of the secondary nitrogen atoms of the BPEI units may be protected.

In another aspect, about 75% to about 99% of the secondary nitrogen atoms of the BPEI units may be protected. In yet another aspect, about 90% to about 95% of the secondary nitrogen atoms of the BPEI units may be protected.

In one aspect, about 90% to 95% of the secondary amino groups in BPEI can be protected, while leaving 80-90% of the primary amino groups unprotected and available for further modification. The density of the free primary amino groups on the surface of BPEI molecule could be further diminished by subsequent blocking, so that a smaller number of them are left free. For example, 3 to 7 [30-70%] of primary amino groups may be left free in the case of BPEI_{1800 D}. The materials obtained at higher protection ranges are more amenable to chemical modification on their remaining free NH groups. This approach is preferable for linking several smaller BPEI molecules due to minimization of loop formation which is unavoidable when using unprotected BPEI. Additionally, in one aspect it may be convenient to attach pendant ligands to the polyethyleneimine units in an one-pot reaction at the same time the cross-linking is accomplished.

It should be noted that any method of selectively protecting nitrogen groups of BPEI units would be considered to be within the scope of the present invention. One exemplary technique is a three-step selective protection technique for small (3-4 N atoms) linear polyamines taught by O'Sullivan et al. 1988 Tetrahedron Letters vol. 29, no. 50, pp 6651-6654 and O'Sullivan et al., 1996 J. Enzyme Inhibition, vol. 11, pp 97-114, both of which are incorporated herein by reference. The technique includes protecting all the primary amino groups as trifluoroacetamides while leaving the secondary amino groups as trifluoroacetate salts, then protecting these secondary amino groups as t-butoxycarbonyl (BOC) or other derivatives, and finally deprotecting the primary amino groups. This technique is sufficiently selective to allow its preparative application with good results in much larger polyamines such as BPEI_{1800D} with about 20 secondary NH's and about 10 primary NH₂'s. If desired, some of the remaining primary amino groups on the exterior of a more or less spherical BPEI (about 10 per BPEI_{1800D} molecule) can be further protected (statistically), leaving an even smaller number of free primary amino groups per poly(alkylene imine) molecule.

Reaction of such protected units with auxiliary ligands (for example, lipids, optional fluorescent tags) further limits the number of available primary amino groups and spaces them further apart, so that their interaction with a bifunctional linker does not lead to intramolecular cross-linking, which can result in gel formation.

The size, i.e., molecular weight, and degree of cross-linking of the cross-linked branched poly(alkylene imine) can be adjusted as desired. The size of the cross-linked polymer will depend on the size or molecular weight of the starting BPAI, the size of the linker, the extent of cross-linking, etc.

Suitable cross-linked branched poly(alkylene imines) of the invention have average molecular weights ranging from about 500, more preferably about 600, to about 25000 Daltons. Particular cross-linked products have average molecular weights ranging from about 4000-20,000 daltons. Still other cross-linked products have average molecular weights ranging from 8000-15,000 daltons.

Short chain linkers are utilized to cross-link the branched polymeric units according to aspects of the present invention. A short chain linker is a group with a backbone length of from about 6 to about 40 atoms, usually but not necessarily symmetrical, which contains at least one biodegradable bond in its backbone. Typical linkers have average molecular weights ranging from about 100 to about 500 Daltons. The precursor molecule to the linker group possesses active chemical groups at each end of its backbone, and these chemical groups may be the same or different. Linking is carried out through these active chemical groups, thereby linking two polyamine units or a polyamine unit and an auxiliary ligand. Furthermore, the linker could be branched, thereby containing three or more terminal active chemical groups. In one aspect such linkers are alkanedioyl groups chains having from 2-20 total carbon atoms in the alkanoyl portion connected via a degradable disulfide bond as in a dithiodialkanoic acid derivative. Such linkers can be represented by the formula:

—C(O)(CH₂)ₓSS(CH₂)_{y}C(O)—

where x and y independently represent integers from 1-12. Such linkers will have amide bonds at their ends connecting the linker to the poly(alkylene imines).

The reactive groups on the precursors to the linkers in the cross-linked products include but are not limited to activated esters such as N-hydroxysuccinimide esters, acyl halides, activated carbonic acid derivatives such as chloroformates, or activated amine derivatives such as isocyanates and isothiocyanates.

The linker may also be a short polyethyleneglycol (“PEG”) group, i.e., a PEG having from about 2-12 oxyethylene groups, containing a biodegradable disulfide bond. Representative reactive groups on the precursors to PEG linkers are terminal activated chemical groups, including but not limited to activated esters such as N-hydroxysuccinimide esters, acyl halides, activated carbonic acid derivatives such as chloroformates, and activated amines such as isocyanates and isothiocyanates.

Depending on the structure chosen for the linker its hydrophilicity/-phobicity could vary, affecting the ease of linker degradation under the biological conditions. This property can be advantageous when fine-tuning of a linked polymer aggregate is desired.

A wide variety of biodegradable bonds are contemplated for incorporation in the short chain linker. In one aspect, for example, the biodegradable bond can include at least one of an ester, an amide, a disulfide, and a phosphate bond. In one specific aspect, the biodegradable bond can be a biodegradable disulfide bond. In another specific aspect, as shown above, a biodegradable disulfide bond can be a part of a diacid moiety, such as an amide of dithiodipropionic acid, or of another dithiodialkanoic acid. One specific example may include a dithiodialkanoic acid with an alkyl chain length from one to 10 carbon atoms. In yet another specific aspect, the biodegradable disulfide linker can include an ethylene glycol moiety having a biodegradable disulfide bond. One non-limiting example of an ethylene glycol moiety is dithiodi(tetraethyleneglycol-carbamate).

Additional non-limiting examples of biodegradable bonds may include esters, amides, phosphates, phosphoesters, hydrazone, cis-asotinyl, and urethane. Since any linker can react in stepwise fashion, the linker can link either different poly(alkylene imine) units or the different areas of the same poly(alkylene imine) unit (loop formation).

The latter will favor the formation of a lightly cross-linked material with poor solubility due to multiple looping, as has been described above. The techniques of the present invention incorporate partial and reversible chemoselective [secondary. vs. primary] blocking/protection of nitrogen atoms in the polymeric units to minimize this problem. Such selective protection facilitates the linking of the polymeric units. This process also allows for convenient incorporation of pendant auxiliary ligands (for example, lipids, or visualizing agents) onto a cross-linked branched poly(alkylene imine).

The ratio of moles of linker to the moles of branched poly(alkylenimine) in the product cross-linked poly(alkylene imine) is from about 0.1:1 to about 5:1. More preferably, the ratio of the moles of linker to the moles of branched poly(alkylenimine) copolymer is from about 1:1 to about 5:1.

In one aspect, the cross-linked branched poly(alkylene imines) of the invention of the invention can be represented by Formula I:

(L_{y}(BPAI))ₓY_{z} I
wherein
BPAI represents a branched polyalkyleneimine unit having a number averaged molecular weight within the range of from about 1000 Daltons to about 25000 Daltons;
Y represents a bifunctional biodegradable linker;
L represents a ligand or functional moiety;
x is an integer in the range from 2 to 20;
y ranges from 0.01 to 100; relating to the statistically averaged degree of incorporation and z is an integer in the range from 1 to 40.

Preferred embodiments of the present invention can be represented by Formula II:

Lₛ[—CO(CH₂)ₐSS(CH₂)ₐCO—]ₚ{[(CH₂)ₙN(—X)—]_{q}}ᵣ II
wherein
L represents a ligand or functional moiety selected from the group consisting of lipids, visualizing agents and targeting ligands;
X represents hydrogen or another —(CH₂) N(X)— branch of the backbone or - in case that the neighboring N atom is also bearing a linker the linker; and
[—CO(CH₂)ₐSS(CH₂)ₐCO—] represents a biodegradable dithiodiacid linker;
“a” is an integer of from 1 to 15;
“n” is an integer of from 2 to 15;
“p” is an integer of from 1 to 100;
“q” is an integer of from 20-500;
“r” is an integer of from 2 to 20; and

“s” is a number from 0.01 to 40, relating to the statistically averaged degree of incorporation.

As has been described, the biodegradable, cross-linked branched poly(alkylene imines) of the invention can be synthesized by cross-linking low molecular weight branched poly(alkylene imines), preferably PEI, units with, for example, a biodegradable disulfide linkage. The resulting biodegradable cross-linked branched poly(alkylene imines) of the are water soluble. Differences in transfection activity between the cross-linked branched poly(alkylene imines) of the invention and that of currently available polymers may be due to the differences in the polymer composition, synthesis scheme and physiochemical properties. The lipid-functionalized cross-linked branched poly(alkylene imines) of the invention have amine groups that are electrostatically attracted to polyanionic compounds such as those found in nucleic acids. These cross-linked branched poly(alkylene imines) condense DNA and form compact structures. In addition, the low toxicity of monomeric degradation products (i.e., the lipid- and linker fragment-bearing low MW BPEIs) after delivery of bioactive materials provides for gene carriers with reduced cytotoxicity and increased transfection efficiency.

As shown in formulae I and II, the biodegradable cross-linked branched poly(alkylene imines) of the invention can also be connected to with various functional moieties or ligands such as tracers (for example, visualizing agents) or targeting ligands either directly or via spacer molecules. In one aspect, only a small portion of the available amino groups is coupled to the ligand. The targeting ligands conjugated to the cross-linked branched poly(alkylene imines) direct the polymer/nucleic acid/drug complex to bind to specific target cells and penetrate into such cells (tumor cells, liver cells, hematopoietic cells, and the like). The target ligands can also be an intracellular targeting element, enabling the transfer of the nucleic acid/drug to be guided towards certain favored cellular compartments (mitochondria, nucleus, and the like).

In one aspect, ligands can include sugar moieties coupled to amino groups of the polymer. Such sugar moieties are preferably mono- or oligo-saccharides, such as galactose, glucose, fucose, fructose, lactose, sucrose, mannose, cellobiose, nytrose, triose, dextrose, trehalose, maltose, galactosamine, glucosamine, galacturonic acid, glucuronic acid, and gluconic acid. The galactosyl unit of lactose provides a convenient targeting molecule for hepatocyte cells because of the high affinity and avidity of the galactose receptor on these cells.

In another aspect, the functional moiety may be a visualizing agent. Visualizing agents include and chromogenic or fluorescent dyes or markers. Although numerous fluorescent markers are contemplated, particular representative examples include rhodamines, Cy3, Cy5, and fluorescein. Furthermore, the molar ratio between the fluorescent marker and the cross-linked branched poly(alkylenimine) may vary depending on the nature intended target and various other procedure details. In certain aspects, the molar ratio of the visualizing agent, e.g., fluorescent marker or chromogenic marker, to the cross-linked branched poly(alkylenimine) is from about from about 0.05 to 1, more preferably about 0.15 to 0.4, and most preferably about 0.25 to 0.35.

Other types of targeting ligands that can be used include peptides such as antibodies or antibody fragments, cell receptors, growth factor receptors, cytokine receptors, folate, transferrin, epidermal growth factor (EGF), insulin, asialoorosomucoid, mannose-6-phosphate (monocytes), mannose (macrophage, some B cells), Lewis^{X} and sialyl Lewis^{X} (endothelial cells), N-acetyll actosamine (T cells), galactose (colon carcinoma cells), and thrombomodulin (mouse lung endothelial cells), fusogenic agents such as polymixin B and hemaglutinin HA2, lysosomotrophic agents, nucleus localization signals (NLS) such as T-antigen, and the like. Furthermore, in one specific aspect, the functional moiety may include a fatty acid group. Non-limiting examples of fatty acid groups are butyroyl, hexanoyl, octanoyl, decanoyl, lauroyl, myristoyl, palmitoyl, stearoyl, myristoleoyl, palmitoleoyl, oleoyl, linoleoyl, alpha-linolenoyl, and combinations thereof.

One advantage of the present invention is that it provides a gene carrier wherein the particle size and charge density are easily controlled. Control of particle size may be important for optimization of a gene delivery system because the particle size often governs the transfection efficiency, cytotoxicity, and tissue targeting in vivo. In one aspect, the particle size may be about 100 nm diameter, which may be an efficient particle size to entry into cells via endocytosis. In another aspect, the particle size may be from about 50 nm to about 300 nm. In another aspect, the particle size may be from about 50 nm to about 500 nm. In addition, positively charged particle surfaces provide for a sufficient chance of binding to negatively charged cell surfaces, followed by entry into cells by endocytosis. The gene carriers disclosed in the present invention have a zeta-potential in the range from about +1 to about +60 mV.

The cross-linked poly(alkylene imines) of the invention are suitable for the delivery of macromolecules such as RNA and DNA into mammalian cells. As has been described, the cross-linked compounds of the invention are particularly suited for the protection and delivery of small nucleotide sequences. The particle size and zeta potential of the cationic polymer/nucleotide complexes can be influenced by the nitrogen to phosphate (N/P) ratio between the polymer and the nucleotide molecules in the polymer/nucleotide complexes. The experiments and results presented below demonstrate that the physico-chemical properties of the biodegradable polymer are compatible with its use as a safe and efficient gene delivery system.

A representative procedure for the preparation of the cross-linked branched poly(alkylene imines) of the invention is shown below in Scheme I. For simplicity, a molecule or unit of branched poly(alkylene imine) (“BPAI”) is represented by a circle with the dots indicating primary nitrogen atoms.

Most of the reactive amino groups, i.e., nitrogen atoms, are protected or blocked prior to cross-linking. In addition to avoiding undesirable reactions with certain nitrogen atoms, protection serves to leave the unprotected amino groups spatially distant from one another, thus hindering formation of intramolecular cross-linking via nitrogen atoms within the same unit.

In the instant process as depicted in Scheme I, primary nitrogen atoms in BPAI are protected first (subsequent to any preliminary reactions with, e.g., a visualizing agent, apart), followed by protection of secondary nitrogens with a different or second protecting group. The former protecting groups are then removed from the primary nitrogen atoms, and those nitrogen atoms can then be reacted with a targeting ligand or a lipophilic group prior to cross-linking. Prior to cross-linking and after the reaction with a lipophilic group etc, a portion of the primary nitrogen atoms are reprotected. The branched, optionally derivatized, branched poly(alkylene imine) is then cross-linked to provide the cross-linked poly(alkylene imine) of the invention. Deprotection of amino groups can then be carried out if desired. The final deprotected cross-linked product is shown as a cyclic 3-unit structure merely as a matter of graphic convention.

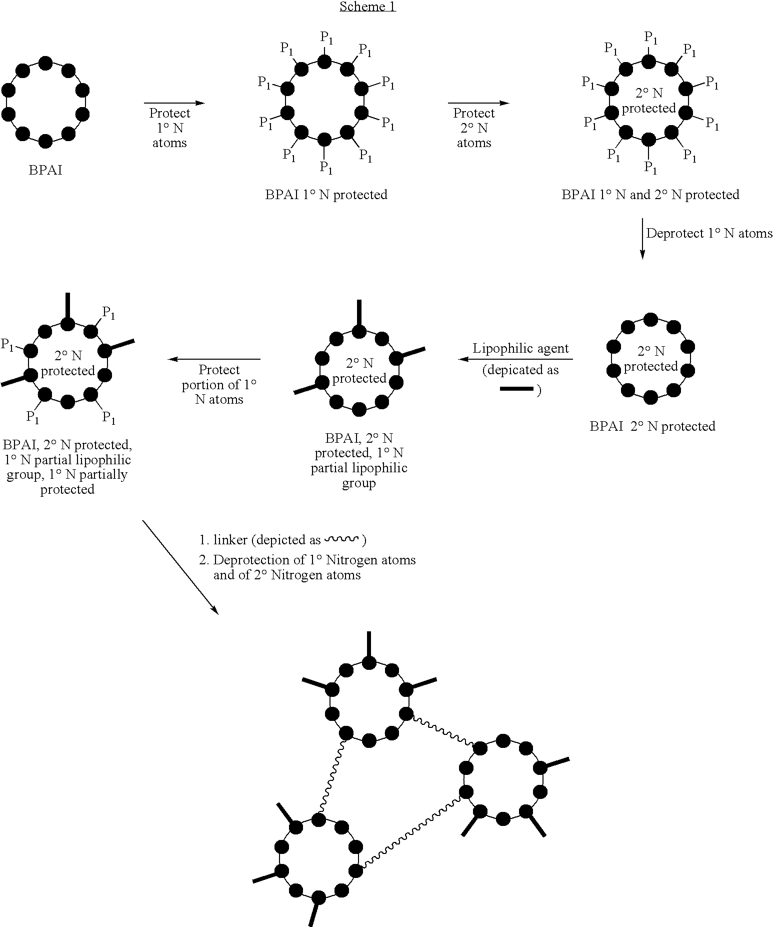

### EXAMPLES

The following examples are provided to promote a more clear understanding of certain embodiments of the present invention, and are in no way meant as a limitation thereon.

### Example 1

### Synthesis of Fluorescently Tagged Selectively Protected (Liss)BPEI_{1800D} (BOC)₂₀

2.4 g (1.33 mmol) of 1800 Da molecular weight BPEI (BPEI_{1800D}) obtained from Polysciences, Inc., Warrington, Pa., USA, are dissolved in 20 ml of dry chloroform, and a solution of 65 mg (ca. 0.1 mmol) of lissamine sulfonylchloride in 10 ml of dry chloroform is added with stirring. The next day the red solution is concentrated under vacuum and the oily residue is taken in 25 ml of acetonitrile. 11 g (77.4 mmol) of ethyl trifluoroacetate and 700 mg (38 mmol) of water are then added to the reaction mixture. The reaction mixture is then stirred and refluxed overnight, and subsequently concentrated in vacuum.

The residue is dissolved in 50 ml of dry THF. 6.5 g (50 mmol) of diisopropylethylamine is added to the solution, followed by 9 g (41.2 mmol) of t-butoxycarbonyl (BOC) anhydride. The stirred reaction mixture is left overnight and then concentrated under vacuum. The viscous residue is dissolved in 150 ml of MeOH; 80 ml of commercial 28% aq. NH₃ solution is added and the stirred mixture is brought to gentle reflux. The next day the mixture is cooled, concentrated under vacuum, and the residue is partitioned between CH₂Cl_{2 [}150 ml] and brine [basified with aq. NH₃ to pH 11]. The aqueous fraction is extracted with CH₂Cl_{2 [}2×50 ml], and the organic fractions are combined, dried over Na₂SO₄ and concentrated under vacuum. NMR analysis of the resulting foam indicates about 20 BOC groups are incorporated per BPEI molecule.

### Example 2

### Synthesis of Selectively Protected BPEI_{1800D} (BOC)₂₀

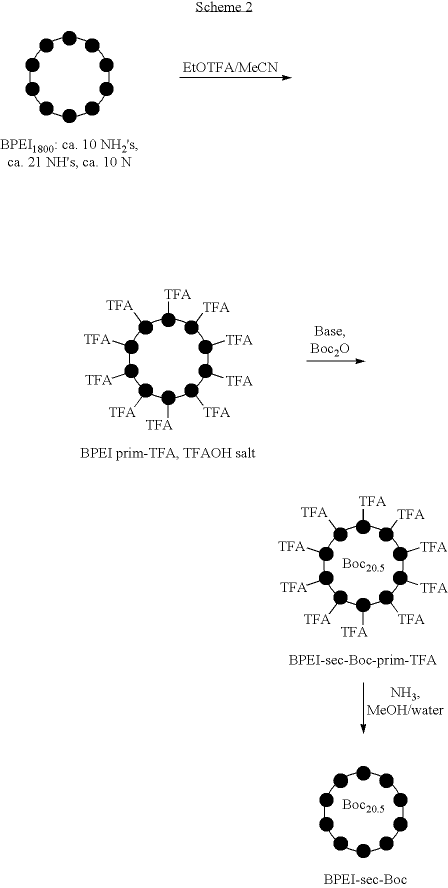

2.4 g (1.33 mmol) of BPEI_{1800D} obtained from Polysciences, Inc., Warrington, Pa., USA, are dissolved in 25 ml of acetonitrile. 11 g (77.4 mmol) of ethyl trifluoroacetate and 700 mg (38 mmol) of water are then added to the reaction mixture. The reaction mixture is then stirred and refluxed overnight, and subsequently concentrated in vacuum. The residue is dissolved in 50 ml of dry THF. 6.5 g (50 mmol) of diisopropylethylamine is added to the solution, followed by 9 g (41.2 mmol) of t-butoxycarbonyl (BOC) anhydride. The stirred reaction mixture is left overnight and then concentrated under vacuum. The viscous residue is dissolved in 150 ml of MeOH; 80 ml of commercial 28% aq. NH₃ solution is added and the stirred mixture is brought to gentle reflux. The next day the mixture is cooled, concentrated under vacuum, and the residue is partitioned between CH₂Cl_{2 [}150 ml] and brine [basified with aq. NH₃ to pH 11]. The aqueous fraction is extracted with CH₂Cl_{2 [}2×50 ml], and the organic fractions are combined, dried over Na₂SO₄ and concentrated under vacuum. NMR analysis of the resulting foam indicates about 20 BOC groups are incorporated per BPEI molecule.

### Example 3

### Preparation of Biodegradable Lipid-Conjugated Cross-Linked BPEI_{1800D} ipid Conjugate

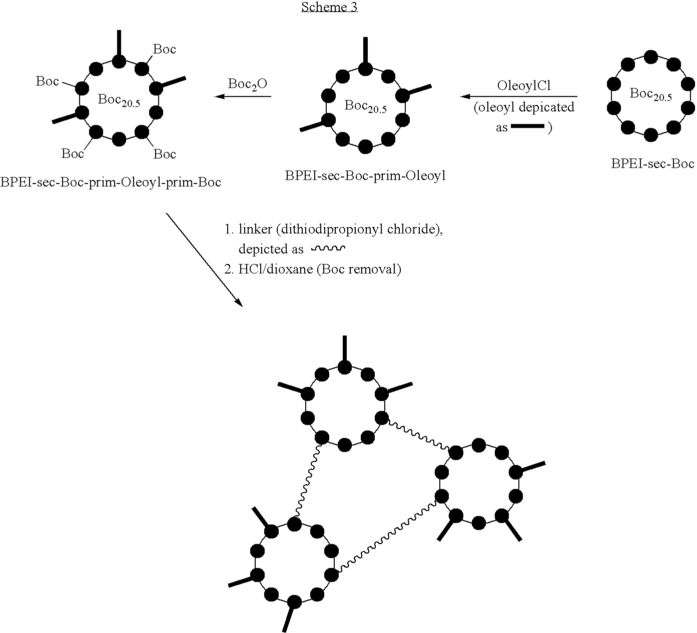

BPEI_{1800D} (BOC)₂₀ (1 g, 262 μMol) made above in Example 2 is dissolved in 3.5 ml CHCl₃ and stirred. Oleoyl chloride (316 mg, 1.05 mMol) is added to the solution. After 1 hr, BOC anhydride (171 mg, 784 μmol) is added and the mixture is stirred. After 24 hours, the mixture is concentrated under vacuum, and the residue is triturated with hexane and dried under vacuum. The resulting foam is taken in 3 ml dry CHCl₃, and a solution of dithiodipropionyl chloride (100 mg in 300 μl CHCl₃, 1.5 eq. to BPEI) obtained from commercial dithiodipropionic acid and thionyl chloride is slowly added with stirring. Cross-linking is allowed to proceed for 48 hours, after which 4M HCl/dioxane (3 ml) is added to remove the BOC protection. After 1 hr the heterogeneous mixture is diluted with ether and centrifuged. The precipitate is 3× repeatedly re-suspended in fresh ether, re-centrifuged, and dried to afford the target material

Schemes 2 and 3 above summarize the synthesis of functionalized cross-linked small BPEI molecules. The circles symbolize BPEI units, the black dots symbolize the primary amino groups in BPEI, the thick lines stand for auxiliary ligands such as oleoyl groups, and wavy lines are used as graphic symbol for dithiodipropionyl linker. BOC is a t-butoxycarbonyl group, TFA is trifluoroacetyl, TFAOH is trifluoroacetic acid.

### Example 3A

### Preparation of Liss-Labeled Biodegradable Lipid-Conjugated Cross-Linked BPEI_{1800D}

The (Liss)BPE_{1800D} (BOC)₂₀ prepared above in Example 1 is cross-linked using essentially the procedure shown above in Example 3 to afford Liss-labeled biodegradable lipid-conjugated cross-linked BPEI_{1800D}.

### Example 4

### Preparation of Water-Soluble Complexes of siRNA with Biodegradable Cross-Linked Branched PEI

This example illustrates the formation of siRNA complexes with the biodegradable cross-linked units of branched PEI. Cross-linked BPEI prepared above in Example 3 is dissolved in sterile water to give a final concentration of 0.01-5 mg/ml. The siRNA is dissolved in sterile water at final concentrations of 0.067-0.33 mg/ml. To make the polymer/siRNA complex, the two components are diluted separately with 5% glucose or 10% lactose or saline to a volume of 1 ml each, and then the siRNA solution is added to the polymer solution at different nitrogen to phosphate ratios (N:P). Complex formation is allowed to proceed for 15 minutes at room temperature.

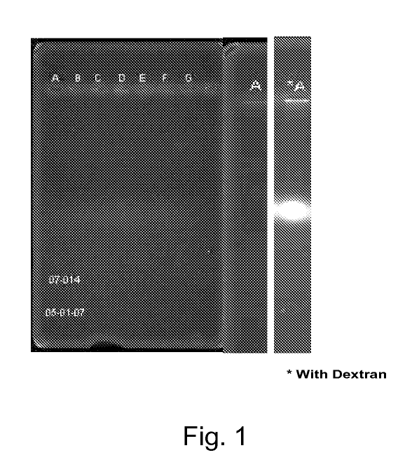

Following complex formation, aliquots are used for measurement of pH, particle size, osmolarity, and zeta potential. The formulation data for polymer/siRNA complexes designed to knockdown glyceraldehyde 3-phosphate dehydrogenase (GAPDH) gene expression is shown in Table 1. To determine the efficiency of complexation, the samples are analyzed by gel electrophoresis. As shown in FIG. 1, complexation with polymer causes a complete cessation of siRNA mobility in the electric field, demonstrating efficient condensation of siRNA by the polymer. The particle size analysis shows siRNA is condensed into ˜150-300 nm particles of positive zeta potential (+25-35 mV) (Table 1). Dextran sulfate (10,000 Dalton) is used to separate the negatively charged siRNA molecules from the positively charged polymer, by displacing the siRNA with negatively charged polymer. This assay is used to conform the electrostatic interaction of the siRNA and the cationic polymer. Additionally, the dextran interaction is reversible and the siRNA is stable following the complexation and the decomplexation events. Furthermore, dextran sulfate is used a measure of the strength of the polymer-nucleic acid interaction.

TABLE 1
Physicochemical Properties of siRNA/polymer
Complexes

| DNA | N:P | Particle size | | Osmolality | Zeta Potential |
|---|---|---|---|---|---|
| (μg) | Ratio | (nm) | pH | (mOsm) | (mv) |
| 20 | 25 | 264 | 4.18 | 304 | 28.29 |
| 50 | 25 | 316 | 3.65 | 308 | 32.85 |
| 100 | 10 | 160 | 3.6 | 223 | 27.86 |
| 20 | 25 | 157 | 4.4 | 317 | 34.24 |
| 50 | 25 | 268 | 3.95 | 325 | 34.80 |
| 100 | 10 | 224 | 4.06 | 224 | 33.99 |

### Example 5

### High siRNA Specificity of the Cross-Linked Branched PEI

This example demonstrates that the use of small molecular weight branched PEI in the biodegradable cross-linked functionalized polymer enhances the polymer efficiency and specificity for siRNA delivery. To further the comparison, cross-linked polymers of linear and separately of branched PEI are complexed with GAPDH siRNA or luciferase plasmid DNA by mixing the DNA or siRNA solutions with that of the polymer solutions at a desirable nitrogen to phosphate ratio (N:P). Cross-linked polymer prepared above in Example 3 is dissolved in sterile water to give a final concentration of 1-5 mg/ml. The siRNA or plasmid DNA is dissolved in sterile water at final concentrations of 0.01-5 mg/ml. To make the polymer/siRNA complex, the polymer solution and the siRNA solution are diluted separately with 5% glucose or saline to a volume of 1 ml each, and then the siRNA solution is added to the polymer solution at a nitrogen to phosphate ratio of 5:1 to 200:1. Complex formation is allowed to proceed for 30 minutes at room temperature. To make the polymer/plasmid DNA complex, the polymer solution and the plasmid DNA solution are diluted separately with 5% lactose to a volume of 1 ml each, and then the plasmid DNA solution is added to the polymer solution at a nitrogen to phosphate ratio of 5:1 to 200:1. Complex formation is allowed to proceed for 30 minutes at room temperature.

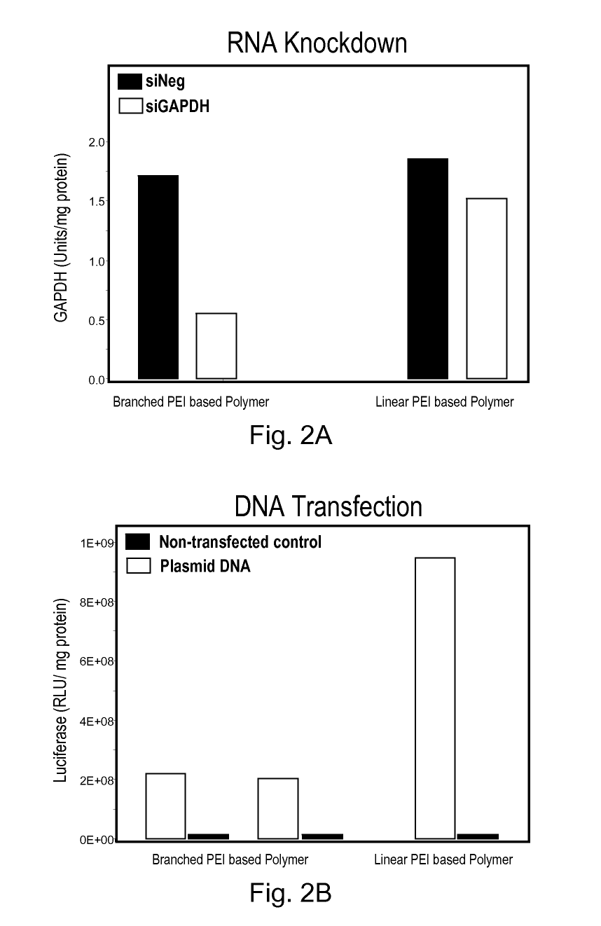

After 30 minutes, DNA complexes are evaluated for luciferase gene transfer while siRNA complexes are evaluated for GAPDH gene knockdown in murine squamous cell carcinomas (SCCVII). SCCVII cells (1.5×10⁵) are seeded to 80% confluence in 12-well tissue culture plates in 10% fetal bovine serum (FBS). Nucleic acid complexes containing 1 μg of luciferase plasmid DNA, 1 μg GAPDH siRNA, or 1 μg of control siRNA (non-targeted sequences) are added into each well in the absence of 10% FBS for 6 hours in a CO₂ incubator. The transfection medium is removed and the cells are incubated for 40 hours with 1 ml of fresh DMEM containing 10% FBS. The cells are washed with phosphate-buffered saline and lysed with TENT buffer (50 mM Tris-Cl [pH 8.0], 2 mM EDTA, 150 mM NaCl, 1% Triton X-100). Luciferase or GAPDH activity in the cell lysate is measured. The final values of luciferase and GAPDH are reported in terms of relative light units (RLU)/mg total protein and units/mg protein, respectively. A total protein assay is carried out using a bicinchoninic acid (BCA) protein assay kit (Pierce Chemical Co., Rockford, Ill.). The results from this experiment are described in FIGS. 2A and 2B. As is shown in FIGS. 2A and 2B, GAPDH or Luciferase activity is compared against appropriate controls. The siRNA complexes prepared with branched PEI based cross-linked polymer produce >90% inhibition of the GAPDH expression while complexes with the linear PEI-based cross-linked polymer produce only marginal inhibition (<20%). In contrast, the efficiency of DNA delivery by linear-PEI-based cross-linked polymer is much higher than that of the branched PEI-based cross-linked polymer. These results demonstrate that the cross-linked branched PEI-based polymer has significantly higher siRNA specificity as compared to that of the cross-linked linear PEI-based polymer.

### Example 6

### Inhibition of VEGF Gene Expression

This example describes the application of a novel cross-linked polymer for vascular endothelial growth factor (VEGF) gene knockdown in cancer cells. VEGF siRNA is complexed with branched PEI cross-linked polymer by mixing the two solutions at nitrogen to phosphate ratios (N:P) of 5:1 and 200:1. Cross-linked BPEI prepared above in Example 3 is dissolved in sterile water to give a final concentration of 0.01-5 mg/ml. The siRNA is dissolved in sterile water at final concentration of 3 mg/ml. To make the polymer/siRNA complex, the polymer solution and the siRNA solution are diluted separately with 5% glucose or saline to a volume of 1 ml each, and then the siRNA solution is added to the polymer solution at a nitrogen to phosphate ratio of 5:1 and 200:1. Complex formation is allowed to proceed for 30 minutes at room temperature.

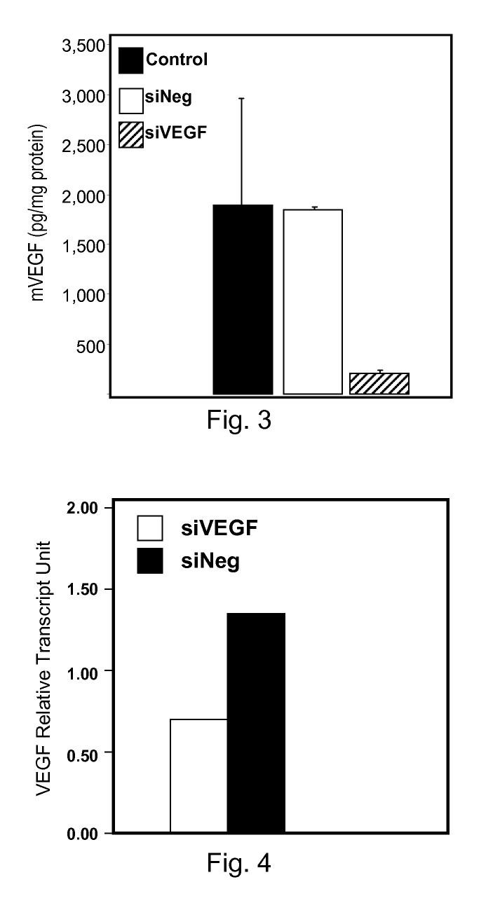

After 30 minutes the siRNA mixture is applied to SCVII cancer cells as described below in order to examine the effect of the mixture on VEGF gene expression. SCVII cells (1.5×10⁵) are seeded to 80% confluence in 12-well tissue culture plates in 10% FBS. siRNA complexes containing 1 μg VEGF siRNA or 0.01 mg/ml of control siRNA (non-targeted sequences) are added into each well in the absence of 10% fetal bovine serum for 6 hours in a CO₂ incubator. The transfection medium is removed and the cells are incubated for 40 hours with 1 ml of fresh DMEM containing 10% FBS. The cells are washed with phosphate-buffered saline and lysed with TENT buffer (50 mM Tris-Cl [pH 8.0], 2 mM EDTA, 150 mM NaCl, 1% Triton X-100). VEGF expression in the cell lysate is quantified by an ELISA. The final values of VEGF are reported in terms of pg/mg total protein and units/mg protein. A total protein assay is carried out using a BCA protein assay kit (Pierce Chemical C, Rockford, Ill.). The results from this experiment are described in FIG. 3. The siRNA complexes prepared with branched PEI based cross-linked polymer produce >90% inhibition of the VEGF expression over the control siRNA complexes.

### Example 7

### Inhibition of VEGF mRNA

This example describes the application of a novel cross-linked polymer for VEGF gene knockdown in cancer cells. VEGF siRNA is complexed with branched PEI cross-linked polymer by mixing the two solutions at nitrogen to phosphate ratios (N:P) of 5:1 to 200:1. Cross-linked BPEI prepared above in Example 3 is dissolved in sterile water to give a final concentration of 1-5 mg/ml. The siRNA is dissolved in sterile water at final concentration of 0.01 mg/ml. To make the polymer/siRNA complex, the polymer solution and the siRNA solution are diluted separately with 5% glucose or saline to a volume of 1 ml each, and then the siRNA solution is added to the polymer solution at a nitrogen to phosphate ratio of 5:1 to 200:1. Complex formation is allowed to proceed for 30 minutes at room temperature.

After 30 minutes the siRNA mixture is applied to SCCVII cancer cells as described below in order to examine the effect of the mixture on VEGF gene expression. SCCVII cells (1.5×10⁵) are seeded to 80% confluence in 12-well tissue culture plates in 10% FBS. siRNA complexes containing 1 μg VEGF siRNA or 0.01 mg/ml of control siRNA (non-targeted sequences) are added into each well in the absence of 10% fetal bovine serum for 6 hours in a CO₂ incubator. The transfection medium is removed and the cells are incubated for 40 hours with 1 ml of fresh DMEM containing 10% FBS. Following the incubation period RNA is purified from the cells using Tri Reagent according to manufactures instructions. Transcript levels are quantified using RTPCR and are reported as relative transcript units. The results from this experiment are described in FIG. 4. The siRNA complexes prepared with branched PEI based cross-linked polymer produced ˜50% inhibition of the VEGF expression over the control siRNA complexes.

### Example 8

### Inhibition of Mouse ApoB mRNA in Liver Cells

This example describes the application of a novel cross-linked polymer for apolipoprotein B (ApoB) gene knockdown in HepG2 liver cells. ApoB siRNA is complexed with cross-linked BPEI prepared above in Example 3 by mixing the two solutions at nitrogen to phosphate ratios (N:P) of 5:1 and 200:1. The cross-linked BPEI is dissolved in sterile water to give a final concentration of 1-5 mg/ml. The siRNA is dissolved in sterile water at final concentration of 0.01 to 5 mg/ml. To make the polymer/siRNA complex, the polymer solution and the siRNA solution are diluted separately with 5% glucose or saline to a volume of 1 ml each, and then the siRNA solution is added to the polymer solution at a nitrogen to phosphate ratio of 5:1 and 200:1. Complex formation is allowed to proceed for 30 minutes at room temperature.

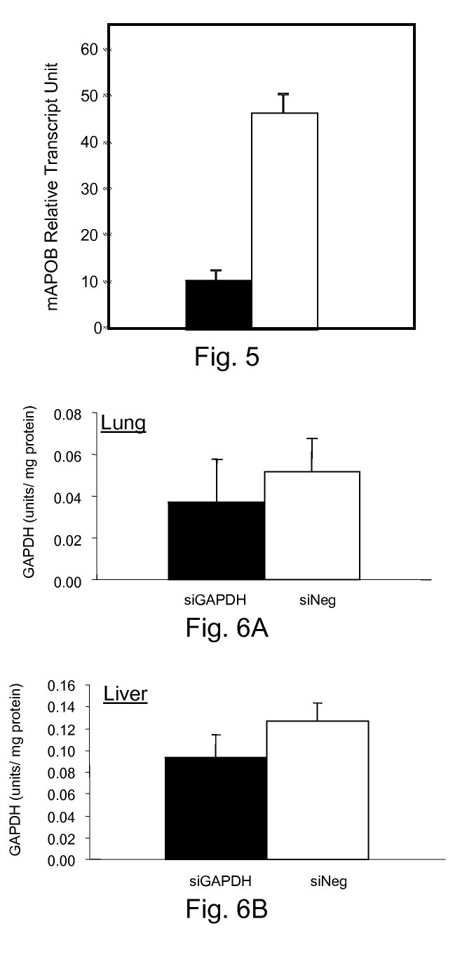

After 30 minutes, the siRNA mixture is applied to HepG2 liver cells as described below in order to measure ApoB gene transcript. HepG2 cells (1.5×10⁵) are seeded to 80% confluence in 12-well tissue culture plates in 10% FBS. siRNA complexes containing 1 μg ApoB siRNA or 0.01 mg/ml control siRNA (non-targeted sequences) are added into each well in the absence of 10% fetal bovine serum for 6 hours in a CO₂ incubator. The transfection medium is removed and the cells are incubated for 40 hours with 1 ml of fresh DMEM containing 10% FBS. The cells are washed with phosphate-buffered saline and lysed with TENT buffer (50 mM Tris-Cl [pH 8.0], 2 mM EDTA, 150 mM NaCl, 1% Triton X-100). ApoB mRNA levels in the cell lysate are quantified by RTPCR and final values are reported in terms of relative transcript units. The results from this experiment are described in FIG. 5. The siRNA complexes prepared with branched PEI based cross-linked polymer produce ˜80% inhibition of the ApoB transcript over the control siRNA complexes.

### Example 9

### Protein Knockdown of Endogenous GAPDH following IV Injection of siRNA Formulated with Cross-Linked BPEI:DOPE

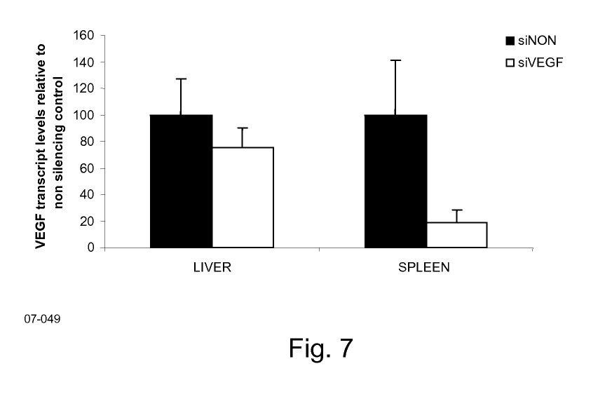

Protein levels of GAPDH are determined in lung and liver tissue of mice 24 hours after the injection of 100 μg GAPDH siRNA. The siRNA is formulated at a 5:1 to 200:1 N:P ratio in 300 μl total volume of 5% glucose, 10% lactose or saline and injected into the tail vein of mice. In this example BPEI prepared above in Example 3, is co-formulated with DOPE at (1:1) (mole: mole) in a liposome form. DOPE is added to promote the release of transfection complexes of cross-linked BPEI/siRNA complexes from the endosomes. After 24 hours mice are euthanized and tissues rapidly removed and frozen in LN₂. The levels of GAPDH are determined in tissue using a commercially available assay, as is shown in FIGS. 6A and 6B. Results indicate that, in both the lung and liver, a 25-30% decrease in GAPDH levels is achieved compared to the GAPDH levels in control mice that are injected with formulated non silencing siRNA. From these studies it can be concluded that siRNA formulated with the lipid-bearing cross-linked BPEI:DOPE delivery systems has the ability to modulate protein expression levels of a highly expressed endogenous gene in multiple tissues following a single IV administration.

### Example 10

### IV Delivery of Lipid-Bearing Cross-Linked BPEI:DOPE Formulated siRNA to Tumors in Lung and Livers to Inhibit Tumor Growth and Metastasis by Knockdown of Endogenous VEGF Gene

Protein levels of VEGF is determined in lung and liver tissue of mice 24 hours after the injection of 100 μg VEGF siRNA. The VEGF siRNA or control siRNA, both formulated with cross-linked material of Examples 3 at a 5:1 to 200:1 N:P ratio in 300 μl total volume, are injected into the tail vein of mice. After 24 hours mice are euthanized and tissues rapidly removed and frozen in LN2. For analysis the frozen tissue is thawed and homogenized in lysis buffer. Protein analysis is by mouse VEGF ELISA (R&D Systems, Minneapolis, Minn.) and normalized to total protein determined using BCA protein assay kit. In an additional study mice are first injected IV with tumor cell line RENCA (renal cell carcinoma) or BL16 (murine melanoma) to establish an animal model of metastatic disease. Approximately 5 days after tumor implant the animals are administered formulated VEGF siRNA or control siRNA as previously described. At time points subsequent to siRNA injection lungs are harvested and VEGF protein and transcript expression levels are determined. The lungs from some animals are used for quantitative determinations of tumor nodules and VEGF expression levels specifically in tumors as measures of the efficacy of formulated siRNA administration.

### Example 11

### IV or Hepatic Portal Vein Administration of Cross-Linked BPEI:DOPE Formulated siRNA for Delivery to Liver Infected with a Single-Stranded, Positive Sense RNA Virus in the Family Flaviviridae Such as Hepatitis C

Intravenous or intrahepatic portal delivery of cross-linked BPEI:DOPE formulated siRNA to liver infected with a single-stranded Hepatitis C virus to inhibit a viral protein crucial for viral survival in the host. The levels of viral protein are determined in liver and blood at different intervals after the injection of 100-300 μg VEGF siRNA. The viral siRNA or control siRNA are formulated at a 5:1 to 200:1 N:P ratio in 300 μl total volume and injected into the tail vein or hepatic portal vein of mice. After 24 hours mice are euthanized and tissues rapidly removed and frozen in LN₂ before analysis.

### Example 12

### Intra-Cranial Delivery of Cross-Linked BPEI:DOPE Formulated RNAi to Inhibit Growth of Gliomas and Other Malignancies of the Brain and to Inhibit the Expression of Aberrant Proteins Associated with Other Disease States (Such as Huntington's Disease)

The effect of local delivery of siRNA, mircoRNA, synthetic shRNA or plasmid encoding for shRNA designed to target a tumor-associated gene or an aberrant gene involved in neurological disorders such as Huntington disease is complexed with cross-linked BPEI:DOPE and administered locally (intracranially) by a single injection or by continuous delivery at the disease site. The injected tissues are analyzed for the efficiency of gene knockdown at various time intervals.

### Example 13

### Delivery of Cross-Linked BPEI:DOPE Formulated siRNA into Solid Tumors Such as Melanoma and Tumors of the Head and Neck to Inhibit Tumor Growth and Metastasis

The effect of local administration of siRNA/cross-linked BPEI:DOPE complexes on the growth of subcutaneously implanted tumors is examined. 4×10⁵ SCCVII cells in 100 μl are implanted subcutaneously on the right flank of female Female CH₃ mice (6-9 weeks, 17-22 grams). The siRNA complexes at a 25:1 N:P ratio are administered locally into the tumors at a siRNA dose of 100-300 μg in an injection volume of 20-60 μl up to three times per week for four weeks starting ˜11 days after tumor implantation. Some of the tumors are harvested at various times after siRNA administration to monitor targeted transcript levels. Additionally, tumor growth is monitored is monitored twice per week using calliper measurement to determine efficacy of formulated siRNA administration.

### Example 14

### Intra-Articular Delivery of Cross-Linked BPEI:DOPE Formulated RNAi In Order to Inhibit Proteins Associated with Join Inflammation, Extracellular Matrix Degradation and Bone Catabolism

The ability to administer formulated siRNA intra-articularly for the treatment of diseases of the joint is examined. For these studies, rats are injected (under anesthesia) intra-articularly (IA) into the right and left knees with up to 100 μg cross-linked BPEI:DOPE formulated siRNA, mircoRNA, synthetic shRNA, or plasmid encoding for shRNA in a total volume of 100 μl. One day following injection, animals are sacrificed and tissues of the joint are harvested and analyzed for targeted transcript and protein levels. Additionally in some studies a model of osteoarthritis will be established. In this model osteoarthritis is surgically induced by performing a medial meniscectomy along with transection of the ligaments. Following a 4 week recovery up to 250 formulated siRNA is injected IA two times/week. At the termination of the study the animals are euthanized, treated knees were harvested and prepared for histopathology and immunohistological analysis using standard procedures in order to evaluate targeted protein and expression levels and efficacy of treatment.

### Example 15

### Delivery of Cross-Linked BPEI:DOPE Formulated RNAi into Intra-Ocular Spaces in Order to Inhibit the Expression of Proteins Associated Chronic Diseases of the Eyes for Example Growth Factors Associated with Angiogenesis

For intraocular injection rats are anesthetized, and the eyes are injected with up to 5 μl of N3-Oleoyl4:DOPE formulated siRNA, mircoRNA, synthetic shRNA or plasmid encoding for shRNA corresponding to VEGF protein. Injection is via a microsyringe using a 29-gauge needle. Eyes will be harvested at various times after injection for determinations of VEGF protein and transcript levels. Additionally standard methods are used for quantification of retinal neovascularization.

### Example 16

### Intrathecal Delivery of Cross-Linked BPEI:DOPE Formulated RNAi to Inhibit Transcripts Involved with Viral Replication and Infection and Transcripts that are Associated with Chronic Pain

For intrathecal delivery rats, are implanted with intrathecal (i.th.) catheters and allowed to recover from surgery prior to treatment. Up to 10 μl of cross-linked BPEI:DOPE formulated siRNA, mircoRNA, synthetic shRNA or plasmid encoding for shRNA is delivered to the lumbar region of the spinal cord via the i.th. catheters. Injections are given up to three times/week. Target protein and transcript expression levels are determined from the lumbar dorsal spinal cord.

### Example 16

### VEGF Transcript Knockdown in Liver and Spleen following Intravenous Injection of VEGF siRNA Formulated with Cross-Linked BPEI

In this example a siRNA targeting murine VEGF was formulated with cross-linked BPEI prepared in Example 3 at a 10:1 N:P ratio in saline. A volume of 300 μl (at a final siRNA concentration of 0.3 mg/ml) was injected into the tail vein of ICR mice. Twenty-four hours after iv administration the animals were euthanized and livers and spleens were harvested for transcript analysis by RTPCR. Results from this study indicate that administration of the VEGF siRNA resulted in a 20% decrease in VEGF transcript relative to the non-silencing control group in the liver and an ˜80% decrease in VEGF transcript in the spleen (FIG. 7).

It is to be understood that the above-described embodiments are only illustrative of the applications of the principles of the present invention. Numerous modifications and alternative embodiments can be derived without departing from the spirit and scope of the present invention and the appended claims are intended to cover such modifications and arrangements. Thus, while the present invention has been shown in the drawings and fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

What is claimed is:

1. A cross-linked poly(alkylene imine) consisting of branched poly(alkylene imine) units having primary, secondary and tertiary amino groups, the units being covalently cross-linked to one another by primary amino groups in the poly(alkylene imine) units and short chain linkers having a biodegradable bond, where
at least one primary amino nitrogen is optionally protected, and
at least one unit is optionally bonded to a targeting ligand, a visualizing agent, and/or a lipophilic group.

2. A cross-linked poly(alkylene imine) according to claim 1, having an average molecular weight of from about 500 to about 25000 Daltons.

3. A cross-linked poly(alkylene imine) according to claim 1, wherein the linkers have an average molecular weight of from about 100 to about 500 Daltons.

4. A cross-linked poly(alkylene imine) according to claim 1, wherein the ratio of the moles of linker to the moles of branched poly(alkylenimine) is from about 1:1 to about 5:1.

5. A cross-linked poly(alkylene imine) according to claim 1, wherein at least one unit is bonded to a targeting ligand, a visualizing agent, and/or a lipophilic group.

6. A cross-linked poly(alkylene imine) according to claim 1, wherein the visualizing agent is a fluorescent or chromogenic markers.

7. A cross-linked poly(alkylene imine) according to claim 1, wherein a plurality of the poly(alkylene imine) units carry lipophilic groups.

8. A cross-linked poly(alkylene imine) according to claim 1, wherein the targeting ligand is a receptor ligand, membrane permeating agent, endosomolytic agent, nuclear localization sequence, or a pH sensitive endosomolytic peptide.

9. A cross-linked poly(alkylene imine) according to claim 7, wherein the lipophilic groups are fatty acid groups selected from the group consisting of butyroyl, caproyl, capryloyl, caproyl acid, lauroyl, myristoyl, plamitoyl, stearoyl, myristoleoyl, palmitoleoyl, oleoyl, linoleoyl, alpha-linolenoyl, and combinations thereof.

10. A cross-linked poly(alkylene imine) according to claim 6, wherein the visualizing agent is selected from the group consisting of rhodamines, Cy3, Cy5, fluorescein, and combinations thereof, and wherein the ratio of moles of poly(alkylene imine) units to moles of visualizing agent is from about 5:1 to about 1000:1.

11. A cross-linked poly(alkylene imine) according to claim 1, wherein the biodegradable bond is an ester, an amide, a disulfide, or a phosphate bond.

12. A cross-linked poly(alkylene imine) according to claim 11, wherein the biodegradable bond is a biodegradable disulfide bond.

13. A cross-linked poly(alkylene imine) according to claim 11, wherein the biodegradable bond is a biodegradable disulfide bond of a dithiodiacid selected from the group consisting of a dithiodialkanoyl acid where the alkanoyl portion has from 2-10 carbon atoms, a biodegradable disulfide bond contained in an ethylene glycol moiety, dithio bis-alkyl diisocyanate, dithio bis-alkyl diisothiocyanate, dithio bis-ethyleneglycolic diisocyanate, and dithio bis-ethyleneglycolic diisothiocyanate.

14. A cross-linked poly(alkylene imine) according to claim 13, wherein the ethylene glycol moiety is dithiodi(tetraethyleneglycol-carbamate).

15. A cross-linked poly(alkylene imine) according to claim 1, wherein the branched poly(alkylenimine) units are branched poly(ethylenimine) units.

16. A compound which is branched poly(alkylene imine) having substantially all of its primary amino nitrogen atoms protected by first protecting groups, and substantially all of its secondary amino nitrogen atoms protected by second protecting groups.

17. A compound which is branched poly(alkylene imine) having substantially all of its primary amino nitrogen atoms unprotected and substantially all of its secondary amino nitrogen atoms protected.

18. A compound which is branched poly(alkylene imine) having a plurality of primary and secondary nitrogen atoms, wherein
(a) substantially all of the secondary amino nitrogen atoms are protected by protecting groups;
(b) the primary amino nitrogen atoms are
(i) unprotected; or
(ii) protected; or
(iii) bonded to R₁, where R₁ is a lipophilic group, a targeting ligand, and/or a visualizing agent; and
at least one of the primary nitrogens is protected, and at least one of the primary nitrogen atoms is bonded to R₁.

19. A pharmaceutical composition comprising a cross-linked poly(alkylene imine) according to claim 1 and a small RNA molecule.

20. A pharmaceutical composition according to claim 19, wherein the small RNA molecule is associated with the cross-linked poly(alkylene imine).

21. A pharmaceutical composition according to claim 19, wherein the small RNA molecule is selected from the group consisting of siRNA, shRNA, dsRNA, ssRNA, mRNA, rRNA, microRNA, and combinations thereof.

22. A pharmaceutical composition according to claim 19 wherein the branched poly(alkylene imine) units are branched poly(ethylenimine) units, and the short chain linker is selected from the group consisting of dithiodialkanoyl acids where the alkanoyl portion has from 2-10 carbon atoms, a biodegradable disulfide bond contained in an ethylene glycol moiety, dithio bis-alkyl diisocyanate, dithio bis-alkyl diisothiocyanate, dithio bis-ethyleneglycolic diisocyanate, and dithio bis-ethyleneglycolic diisothiocyanate; and
a small RNA molecule.

23. A pharmaceutical composition according to claim 19, wherein the small RNA molecule is associated with the cross-linked poly(alkylene imine).

24. The polymeric nucleotide delivery composition of claim 19, wherein the small RNA molecule is selected from the group consisting of siRNA, shRNA, dsRNA, ssRNA, mRNA, rRNA, microRNA and combinations thereof.

25. A pharmaceutical composition comprising:
a cross-linked poly(alkylene imine) according to claim 15 and a nucleotide molecule.

26. A pharmaceutical composition according to claim 25, wherein the nucleotide molecule is associated with the cross-linked poly(alkylene imine).

27. A pharmaceutical composition according to claim 26, wherein the nucleotide molecule is selected from the group consisting of siRNA, shRNA, dsRNA, ssRNA, mRNA, rRNA, microRNA, DNA, plasmids, cDNA, and combinations thereof.

28. A pharmaceutical composition according to claim 25, wherein the molar ratio of nitrogen in the poly(alkylene imine) units to phosphate in the nucleotide molecule is from about 5:1 to about 200:1.

29. A pharmaceutical composition according to claim 25, further comprising a coformulant selected from the group consisting of dioleoyl phosphatidylethanolamine, cholesterol, galactosylated lipid, polyethyleneglycol-conjugated lipid, and combinations thereof.

30. A process for making a cross-linked poly(alkylene imine) according to claim 1 the process comprising:
(a) reversibly blocking at least about 50%, of secondary nitrogen atoms within branched poly(alkylenimine) to form protected branched poly(alkylenimine); and
(b) cross-linking the protected branched poly(alkylenimine) with a short-chain linker having a biodegradable bond, and

31. A process according to claim 30, further comprising (c) deprotecting the protected branched poly(alkylenimine) units following cross-linking.

32. A process according to claim 30, wherein in (a) from about 75% to about 99% of the secondary nitrogen atoms of the branched poly(alkylenimine) are reversibly blocked.

33. A process according to claim 30, wherein in (a) from about 90% to about 95% of the secondary nitrogen atoms of the branched poly(alkylenimine) are reversibly blocked.

34. A process for making a cross-linked poly(alkylene imine) according to claim 1 comprising:
(a) reversibly blocking at least about 75% of the primary nitrogen atoms within branched poly(alkylene imine) to form a primary-nitrogen protected branched poly(alkylenimine);
(b) reversibly blocking at least about 50%, of secondary nitrogen atoms within the primary-nitrogen branched poly(alkylenimine) to form primary-nitrogen and secondary-nitrogen protected branched poly(alkylenimine);
(c) deprotecting the primary nitrogen atoms in the primary-nitrogen and secondary-nitrogen protected branched poly(alkylenimine) to produce secondary-nitrogen protected branched poly(alkylenimine); and
(d) cross-linking the secondary-nitrogen protected branched poly(alkylenimine) with a short-chain linker having a biodegradable bond to form a cross-linked branched poly(alkylenimine).

35. A process according to claim 34, further comprising (e) removing the protecting groups from the cross-linked branched poly(alkylenimine) following cross-linking.

36. A process according to claim 34, further comprising (c1) reacting the secondary-nitrogen protected branched poly(alkylenimine) with a targeting ligand, a visualizing agent, and/or a lipophilic group prior to cross-linking.

37. A process according to claim 34, further comprising (1a) reacting the branched poly(alkylenimine) with a targeting ligand, a visualizing agent, and/or a lipophilic group prior to protecting either the primary or secondary nitrogen atoms.

38. A process according to claim 34, further comprising (a1) reacting an excess of the branched poly(alkylenimine) with a visualizing agent prior to protecting either the primary or secondary nitrogen atoms.

39. A cross-linked branched poly(alkylene imine) prepared by the process of claim 30.

40. A cross-linked branched poly(alkylene imine) prepared by the process of claim 34.
